(19) United States
(12) Patent Application Publication
(10) Pub. No.: US 2014/0140928 A1
(43) Pub. Date: May 22, 2014

(54) 5HT1A ANTAGONIST USEFUL FOR IN VIVO IMAGING

(71) Applicant: Ian Martin Newington, Buckinghamshire (GB)

(72) Inventor: Ian Martin Newington, Buckinghamshire (GB)

(73) Assignee: GE HEALTHCARE LIMITED, BUCKINGHAMSHIRE (GB)

(21) Appl. No.: 14/131,486

(22) PCT Filed: Jul. 27, 2012
(86) PCT No.: PCT/EP2012/064798

§ 371 Date: Jan. 8, 2014

Related U.S. Application Data
(60) Provisional application No. 61/512,450, filed on Jul. 28, 2011.

(30) Foreign Application Priority Data
Jul. 28, 2011 (GB) 1112987.1

(51) Int. Cl. A61K 51/04; C07D 213/75

(52) U.S. Cl. CPC A61K 51/0459; C07D 213/75
USPC 424/1.89; 544/360; 422/554

(57) ABSTRACT

The present invention provides a novel compound of formula (I) useful for in vivo imaging of 5-HT_{1 A} receptors in a subject. Also provided by the present invention is a precursor compound useful in the preparation of the compound of the invention, as well as said method of preparation. The present invention additionally provides methods for the use of the compound of the invention in an in vivo imaging method, and use of that in vivo imaging method in diagnosis and therapy monitoring.

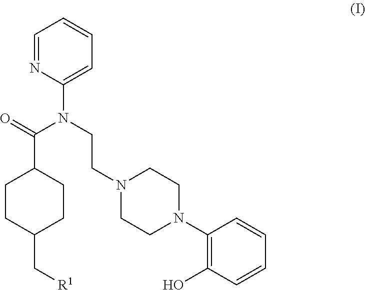

### TECHNICAL FIELD OF THE INVENTION

The present invention relates to radiodiagnostic compounds and precursors thereof, methods of making those compounds, and methods of their use as imaging agents for a serotonin receptor (e.g., the 5-HT_{1A} receptor). The radiodiagnostic compounds of the invention preferably have high affinity for said serotonin receptor and are suitable for use in the in vivo imaging techniques positron-emission tomography (PET) or single-photon emission computed tomography (SPECT), and preferably in PET. Pharmaceutical compositions comprising an imaging-effective amount of radiolabeled compounds are also disclosed. The present invention also relates to non-radiolabeled compounds, methods of making those compounds, and methods of use thereof to treat various neurological and/or psychiatric disorders.

### DESCRIPTION OF RELATED ART

Serotonin (5-hydroxytryptamine; 5-HT) plays a role in several neurological and psychiatric disorders. It has been variously linked with major depression, bipolar disorder, eating disorders, alcoholism, pain, anxiety, obsessive-compulsive disorders, Alzheimer's disease, Parkinson's disease and other psychiatric illnesses. It is also involved in mediating the action of many psychotropic drugs including antidepressants, antianxiety drugs and antipsychotics. There are more than a dozen known subtypes of serotonin receptors. Among these serotonin receptors, 5-HT_{1A} receptors play a role as a presynaptic autoreceptor in the dorsal raphe nucleus and as a postsynaptic receptor for 5-HT in terminal field areas. The serotonin system in the brain is an important neurotransmission network regulating various physiological functions and behaviour including anxiety and mood states. (See Rasmussen et al Chapter 1 “Recent Progress in Serotonin 5HT_{1A} Receptor Modulators”, in Annual Reports in Medicinal Chemistry, Vol. 30, Section I, pp. 1-9, 1995, Academic Press, Inc.).

WO0016777 discloses that a 5-HT_{1A} receptor agonist, buspirone is efficacious in treating a variety of symptoms associated with ADHD (attention deficit hyperactivity disorder), and that combined use of a D2 receptor agonist and 5-HT_{1A} provides effective treatments for ADHD and Parkinson's disease.

5-HT_{1A} agonists are effective in the treatment of cognitive impairment in Alzheimer's disease, Parkinson's disease or senile dementia. U.S. Pat. No. 5,824,680 discloses that a 5-HT_{1A} agonist, ipsapirone, is effective in treating Alzheimer's disease by improving memory. U.S. Pat. No. 4,687,772 describes that a 5-HT_{1A} partial agonist, buspirone, is useful for improving short term memory in patients in need of treatment. WO9304681 discloses that use of 5-HT_{1A} partial agonists have been used for the treatment or prevention of cognitive disorders associated with Alzheimer's disease, Parkinson's disease or senile dementia.

5-HT_{1A} agonists are also effective in the treatment of depression. U.S. Pat. No. 4,771,053 describes that a 5-HT_{1A} receptor partial agonist, gepirone, is useful in alleviation of certain primary depressive disorders, such as severe depression, endogenous depression, major depression with melancholia, and atypical depression. WO0152855 disclose that the combined use of the 5-HT_{1A} receptor partial agonist gepirone with an antidepressant can effectively treat depression.

However, the aforementioned patents and publications do not utilize radioligands.

The most successful radioligands studied so far for 5-HT_{1A} receptors are antagonists tracers which bind with both the G-protein-coupled high affinity (HA) state and uncoupled low affinity (LA) state of 5-HT_{1A} receptors disclosed in U.S. Pat. No. 6,056,942. U.S. Pat. No. 6,056,942 describes selective 5-HT_{1A} antagonists radiolabelled with ³H or ¹¹C ligands which are useful, for example, in pharmacological screening procedures and in PET studies. In contrast, agonists bind preferentially to the HA state of the 5-HT_{1A} receptor.

There have only been a few studies performed on select 5-HT_{1A} agonist radiotracers in a living brain. These studies unfortunately have resulted in low radiochemical yield (less than 2%) and purity (WO2009006227). Thus, there is still a need in the art for radiolabeled serotonin receptor agonist, partial agonist, inverse agonist, or antagonist modulators that are highly selective for imaging 5-HT_{1A} receptors. There also remains a need in the art for selective radioactive tracers, which are useful for imaging 5-HT_{1A} receptors in vivo by powerful imaging methods like PET or SPECT. There is also a need for a more efficient method of obtaining these selective radioactive tracers that yields a higher radiochemical yield and purity.

### SUMMARY OF THE INVENTION

The present invention provides a novel compound useful for in vivo′imaging of 5-HT_{1A} receptors in a subject. The compound of the invention has a better pharmacological profile and is more readily radiolabeled than other known desmethyl WAY-like analogues. Also provided by the present invention is a precursor compound useful in a method to prepare the compound of the invention, with said method to prepare providing an additional aspect of the invention. The present invention additionally provides methods for the use of the compound of the invention in an in vivo imaging method, as well as use of that in vivo imaging method for diagnosis and therapy monitor ng.

### DETAILED DESCRIPTION OF THE INVENTION

### Compound

In one aspect, the present invention provides a compound of Formula I:

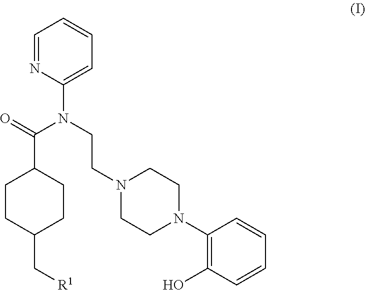

wherein R¹ is an isotope of fluorine.

The term “isotope of fluorine” is intended to encompass stable as well as radioactive isotopes of fluorine, wherein ¹⁹F is a preferred stable isotope of fluorine, and ¹⁸F is a preferred radioactive isotope of fluorine.

The compound of Formula I has high affinity and selectivity for the serotonin (5-HT_{1A}) receptor compared to the other known transporters, receptors, enzymes and proteins; as well as sufficient lipophilicity to allow rapid blood-brain-barrier penetration and generation of polar metabolites that do not cross the blood-brain-barrier.

Although no specific stereoisomers of the compound of Formula I are indicated above, it is understood that the compound exists in all possible stereoisomeric forms, particularly around the cyclohexyl ring. Stereoisomers encompassed by the compound of Formula I include, but are not limited to, compounds of the Formulae Ia and Ib:

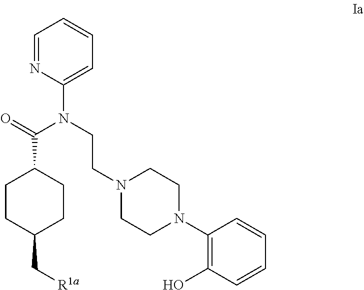

wherein R^{1a} is an isotope of fluorine; and,

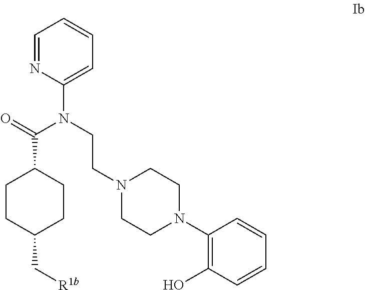

wherein R^{1b} is an isotope of fluorine.

### Pharmaceutical Composition

In a preferred embodiment, the compound of any one of the above Formulae I, Ia and Ib is provided as a pharmaceutical composition comprising said compound and a physiologically acceptable carrier or vehicle.

The present pharmaceutical compositions can be administered orally or by any other convenient route, for example, by infusion or bolus injection, or by absorption through epithelial or mucocutaneous linings (e.g., oral, rectal, and intestinal mucosa, etc.) and can be administered together with another biologically active agent. Administration can be systemic or local. Various delivery systems suitable for administration to a subject are know e.g., encapsulation in liposomes, microparticles, microcapsules, capsules, etc.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result in the release of the compound of the present invention into the bloodstream. The mode of administration is left to the discretion of the practitioner.

In one embodiment, the compound of the present invention is administered orally.

In another embodiment, the compound of the present invention is administered intravenously.

In another embodiment, the compound of the present invention is administered transdermally.

In other embodiments, it can be desirable to administer the compound of the present invention locally. This can be achieved, for example, and not by way of limitation, by local infusion during surgery, by injection, by means of a catheter, by means of a suppository or enema, or by means of an implant, said implant being of a porous, non-porous, or gelatinous material including membranes, such as sialastic membranes, or fibres.

In certain embodiments, it can be desirable to introduce the compound of the present invention into the central nervous system or gastrointestinal tract by any suitable route, including intraventricular, intrathecal, epidural injection, and enema. Intraventricular injection can be facilitated by an intraventricular catheter, for example, attached to a reservoir, such as an Ommaya reservoir.

Pulmonary administration can also be employed, e.g., by use of an inhaler of nebulizer, and formulation with an aerosolizing agent, or via perfusion in a fluorocarbon or a synthetic pulmonary surfactant.

In another embodiment the compound of the present invention can be delivered in a vesicle, in particular a liposome (see Langer, 1990 Science; 249: 1527-1533 and “Liposomes in the Therapy of Infectious Disease and Cancer”, pp. 317-327 and 353-365 (1989)).

In yet another embodiment the compound of the present invention can be delivered in a controlled-release system or sustained-release system (see, e.g., Goodson, in “Medical Applications of Controlled Release”, vol. 2, pp. 115-138 (1984)). Other controlled or sustained-release systems discussed in the review by Langer (1990 Science; 249: 1527-1533) can be used. In one embodiment a pump can be used (Langer, supra; Sefton 1987 CRC Crit Ref Biomed Eng; 14: 201; Buchwald et al 1980 Surgery; 88: 507; and Saudek et al 1989 N Engl J Med; 321: 574).

In another embodiment polymeric materials can be used (see “Medical Applications of Controlled Release”, Langer and Wise, Eds. (1974); “Controlled Drug Bioavailability, Drag Product Design and Performance” Smolen and Ball Eds. (1984); Ranger and Peppas 1983 J Macromol Sci Rev Macromol Chem; 2: 61; Levy et al 1935 Science; 228: 190; During et al 1989 Ann Neural; 25: 351; and Howard et al 1989 J Neurosurg; 71: 105).

The present pharmaceutical compositions can optionally comprise a suitable amount of a physiologically acceptable excipient so as to provide the form for proper administration of the compound of the present invention to the subject. Such a physiologically acceptable excipient can be a liquid, such as water for injection, bactereostatic water for injection, sterile water for injection, and oils, including those of petroleum, subject, vegetable, or synthetic origin, such as peanut oil, soybean oil, mineral oil, sesame oil and the like. The pharmaceutical excipients can be saline, gum acacia; gelatine, starch paste, talc, keratin, colloidal silica, urea and the like. In addition, auxiliary, stabilizing, thickening, lubricating, and colouring agents can be used. In one embodiment the physiologically acceptable excipients are sterile when administered to a subject. Water is a particularly useful excipient when the compound of the present invention is administered intravenously. Saline solutions and aqueous dextrose and glycerol solutions can also be employed as liquid excipients, particularly for injectable solutions. Suitable pharmaceutical excipients also include starch, glucose, lactose, sucrose, gelatine, malt, lice, flour, chalk, silica gel, sodium stearate, glycerol monostearate, talc, sodium chloride, dried skim milk, glycerol, propylene, glycol, water, ethanol and the like. The present pharmaceutical compositions, if desired, can also contain minor amounts of wetting or emulsifying agents, or pH buffering agents. The present pharmaceutical compositions can take the form of solutions, suspensions, emulsion, tablets, pills; pellets, capsules, capsules containing liquids, powders, sustained-release formulations, suppositories, emulsions. aerosols, sprays, suspensions, of any other form suitable for use.

In one embodiment the pharmaceutical composition is in the form of a capsule (U.S. Pat. No. 5,698,155). Other examples of suitable physiologically acceptable excipients are described in Remington's Pharmaceutical Sciences 1447-1676 (Alfonso R. Gennaro, Eds., 19th Ed. 1995).

In one embodiment the compound is formulated in accordance with routine procedures as a pharmaceutical composition adapted for oral administration to human beings. Pharmaceutical compositions for oral delivery can for example be in the form of tablets, lozenges, aqueous or oily suspensions, granules, powders, emulsions capsules, syrups, or elixirs. Orally administered pharmaceutical compositions can contain one or more agents, for example, sweetening agents such as fructose, aspartame or saccharin; flavouring agents such as peppermint, oil of wintergreen, or cherry; colouring agents; and preserving agents, to provide a pharmaceutically palatable preparation. Moreover, where in tablet or pill form, the pharmaceutical compositions can be coated to delay disintegration and absorption in the gastrointestinal tract thereby providing a sustained action over an extended period of time. A time-delay material such as glycerol monostearate or glycerol stearate can also be used. Oral pharmaceutical compositions can include standard excipients such as mannitol, lactose, starch, magnesium stearate, sodium saccharin, cellulose, and magnesium carbonate.

In one embodiment the excipients are of pharmaceutical grade.

In another embodiment the compound can be formulated for intravenous administration. Typically, pharmaceutical compositions for intravenous administration comprise sterile isotonic aqueous buffer. Where necessary, the pharmaceutical compositions can also include a solubilizing agent. Pharmaceutical compositions for intravenous administration can optionally include a local anaesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampule or sachet indicating the quantity of active agent. Where the compound is to be administered by infusion, it can be dispensed, for example, with an infusion bottle containing sterile pharmaceutical grade water or saline. Where the compound is administered by injection, an ampule of sterile water for injection or saline can be provided so that the ingredients can be mixed prior to administration.

The compound can be administered by controlled-release or sustained-release means or by delivery devices that are well known to those of ordinary skill in the art. Examples include, but are not limited to, those described in U.S. Pat. No. 3,845,770, U.S. Pat. No. 3,916,899, U.S. Pat. No. 3,536,809, U.S. Pat. No. 3,598,123, U.S. Pat. No. 4,008,719, U.S. Pat. No. 5,674,533, U.S. Pat. No. 5,059,595, U.S. Pat. No. 5,591,767, U.S. Pat. No. 5,120,548, U.S. Pat. No. 5,073,543, U.S. Pat. No. 5,639,476, U.S. Pat. No. 5,431,922, U.S. Pat. No. 5,354,556, U.S. Pat. No. 5,733,556. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the radiolabeled and non-radiolabeled compounds of the invention. The invention thus encompasses single unit dosage forms suitable for oral administration such as, but not limited to, tablets, capsules, gelcaps, and caplets that are adapted for controlled- or sustained-release. The invention also encompasses transdermal delivery devices, including but not limited to, a transdermal patch and other devices, such as those described in U.S. Pat. No. 5,633,009.

In one embodiment a controlled- or sustained-release composition comprises a minimal amount of a radiolabeled compound to image one or more HA serotonin (5-HT_{1A}) receptors in a subject. Advantages of controlled- or sustained-release pharmaceutical compositions include extended activity of the drug, reduced dosage frequency, and increased subject compliance. In addition, controlled- or sustained-release pharmaceutical compositions can favourably affect the time of onset of action or other characteristics, such as blood levels of the compound, and can thus reduce the occurrence of adverse side effects. Controlled- or sustained-release pharmaceutical compositions can initially release an amount of a compound that promptly produces the desired therapeutic effect, and gradually and continually release other amounts of the compound to maintain this level of therapeutic effect over an extended period of time. To maintain a constant level of the compound in the body, the compound can be released from the dosage form at orate that will replace the amount of radiolabeled compound being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions.

### Methods of Imaging, Diagnosis and Treatment

In another aspect, the present invention provides a method for imaging one or more 5-HT_{1A} receptors in a subject in vivo, the method comprising:
(a) administering to the subject an imaging-effective amount of a compound as defined herein wherein said isotope of fluorine is ¹⁸F; and then,
(b) detecting the radioactive emission of said ¹⁸F.

Administration to said subject is preferably via intravenous administration as a pharmaceutical composition, as described in more detail above. This aspect of the invention can also be understood to start with an alternative step (a) which comprises providing said subject pre-administered with said imaging-effective amount of said compound. The suitable and preferred indications set out for the compound of the invention wherein R¹ is ¹⁸F apply equally to the method for imaging of the invention.

The term “subject” as used herein, includes, but is not limited to, a non-human animal, such as a cow, monkey, horse, sheep, pig, chicken, turkey, quail, cat, dog, mouse, rat, rabbit, or guinea pig; and a human. In one embodiment, a subject is a human.

The term “imaging-effective amount” when used in connection with radiolabeled compounds of the present invention is an amount of the compound that is sufficient to produce a visible image when the compound has been administered to a subject and the radiation emitted by the compound is detected using PET or autoradiography. An imaging-effective amount of the compound of Formula I wherein R¹ is ¹⁸F can be determined using standard clinical and nuclear medicine techniques. In addition, in vitro or in vivo testing can optionally be employed to help identify optimal dosage ranges. The precise dose to be employed will also depend on certain factors—the route of administration, and the identity of the subject—and should be decided according to the judgment of the practitioner and each subject's circumstances in view of, e.g., published clinical studies. The imaging-effective dosage amounts are in the range of about 170 to 380 MBq (megabecquerel) compared to the total amounts administered; that is, if more than one dose of a radiolabeled compound is administered, the imaging-effective dosage amounts correspond to the total amount administered.

A suitable method for imaging in this method is positron emission tomography (PET) given that the isotope of fluorine is ¹⁸F, and the radioactive emission from ¹⁸F is a positron. The detecting step therefore comprises detection of pairs of annihilation (gamma) photons, each of which results when a positron emitted from the ¹⁸F present in said compound interacts with an electron. Detection is carried out by means of a scintillator in the PET scanning device and the detected information is converted into an image. Preferably, said radioactive emission is detected in the brain of said subject.

PET is a dynamic, non-invasive imaging technique used in nuclear medicine to study various biochemical and biological process in vivo. In PET, radiolabeled and non-radiolabeled compounds may be administered in nanomolar or picomolar concentrations, allowing imaging studies to be performed without perturbing the biological system being studied. Like other dynamic imaging protocols, PET has the ability collect images repeatedly over time and provide information about regional distribution of the tracer as well as the change in compartmental distribution as a function of time. As such, PET lends itself directly to measuring kinetic processes, such as rate of tracer uptake by cells, substrate metabolic rates, receptor density/affinity, and regional blood flow.

A PET tracer emits positrons which annihilate with electrons up to a few millimetres away, causing two gamma photons to be emitted in opposite directions. A PET scanner detects these emissions “coincident” in time, which provides more radiation event localization information and thus relatively high resolution images.

In a preferred embodiment, the method for imaging is can ied out on a subject who is known or suspected to have a neurological disorder. A neurological disorder is an affective disorder, an anxiety disorder, an eating disorder, an addictive disorder, a sleep disorder, a disease associated with cognitive dysfunction, a neurodegenerative disease, such as stroke; a seizure disorder, a pain disorder; a panic disorder, a disorder of movement, or an obsessive-compulsive disorder.

Preferably, said disease associated with cognitive dysfunction is Alzheimer's disease.

Preferably, said neurodegenerative disease is stroke.

Preferably, said disorder of movement is Parkinson's disease.

Preferably, said seizure disorder is epilepsy.

Preferably, said affective disorder is depression.

In the method for imaging one or more 5HT_{1A} receptors, said compound preferably selectively binds to the 5-HT_{1A} receptor relative to other serotonin receptors.

In another embodiment of the present invention, a method for diagnosis is provided wherein said method comprises the method for imaging as defined above, wherein said subject is known or suspected to have a neurological disorder, followed by the steps of:
(c) comparing the radioactive emission of ¹⁸F detected for said subject with standard values;
(d) finding any significant deviation between said radioactive emission of ¹⁸F detected for said subject as compared with said standard values;
(e) attributing said deviation to a neurological disorder.

The suitable and preferred indications for the compound provided above in connection with the method for imaging apply equally to the method for diagnosis of the invention.

In a further embodiment, the present invention provides a method for treating a disease associated with abnormal 5-HT_{1A} receptor function comprising administering to the subject in need thereof an effective amount of a compound as defined herein wherein said isotope of fluorine is ¹⁹F.

Another embodiment of the invention comprises a method for treating a neurological disorder in a subject, the method comprising administering to said subject a therapeutically effective amount of a compound as defined herein wherein said isotope of fluorine is ¹⁹F.

The term “effective amount” or “therapeutically effective amount” is an amount that is effective to treat or prevent a disease or disorder as defined herein in a subject, or to stabilize the mood of a subject having a mood disorder.

A yet further embodiment of the present invention is a method for monitoring the effect of treatment of a human or animal body with a drug to combat or treat a condition associated with a neurological disorder, said method comprising said method for imaging as suitably and preferably defined herein, optionally but preferably being effected before, during and after treatment with said drug.

The suitable and preferred indications concerning the subject and neurological disorder, provided above in connection with the method for imaging apply equally to the methods for diagnosis, treatment and monitoring the effect of treatment of the invention.

In an alternative, the present invention provides a compound as defined herein for use in medicine. Preferably, said use in medicine is any one of the methods for imaging, diagnosis, treatment and monitoring the effect of treatment as suitably and preferably described in more detail above.

In another alternative, the present invention provides for the use of a compound of Formula I as defined herein in the manufacture of a pharmaceutical for use in any one of methods for imaging, diagnosis, treatment and monitoring the effect of treatment as suitably and preferably described in more detail above.

### Methods to Obtain Compounds of the Invention

Compounds of Formula I as defined herein may be prepared by adaptation of the method described by Choi et al (2010 Bull Korean Chem Soc; 31(8): 2371-2374) for the preparation of MeFWAY. Reaction of 2-aminopyridine 1 with chloroacetyl chloride 2 at room temperature provides the 2-(chloroacetyl)amidopyridne 3:

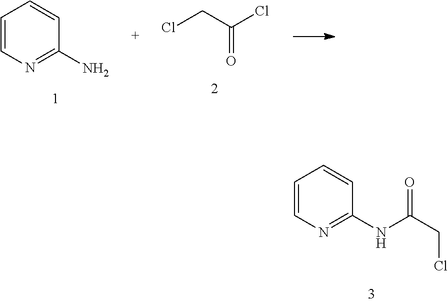 [SMILES: Nc1ccccn1 ; O=C(Cl)CCl ; O=C(CCl)Nc1ccccn1]

In the next step 3 is treated with the phenylpiperzine 4 in DMF at 80° C. in the presence of K₂CO₃ and NaI to give the corresponding phenylpiperazinyl amidopyridine 5:

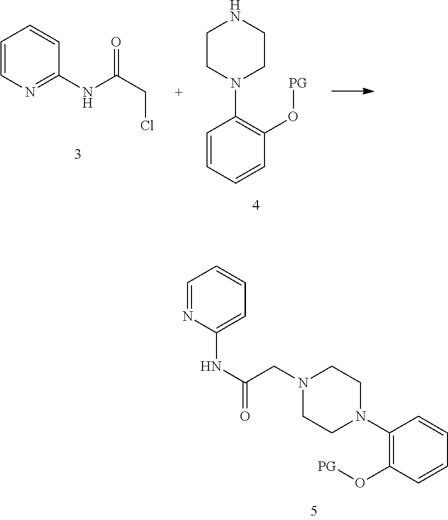

wherein PG represents hydrogen or a protecting group and is preferably a protecting group. A suitable protecting group is a methoxyethoxymethyl (MEM) group, a methoxymethyl (MOM) group, a t-butyldimethylsilyl (TBDMS) group, a trimethylsilyl (TMS) group or a benzyl group such as 4-methoxybenzyl or 2,4-dimethoxybenzyl. Methyl, which takes the place of PG in certain intermediates described below is not to be regarded as a suitable protecting group.

Intermediate 5 where PG is hydrogen might alternatively be arrived at by first making the methylated derivative according to the method of Choi et al (supra), i.e. methyl is present rather than PG, and demethylating to arrive at 5 and adding the protecting group PG if desired. Non-limiting examples of reagents that can be used for this demethylation include BBr₃, trimethylsilyliodide, pyridinium tosylate and potassium t-butylthiolate.

5 is then reduced to obtain 6, a derivative of the known selective antagonist for 5HT1a receptors, WAY-100634:

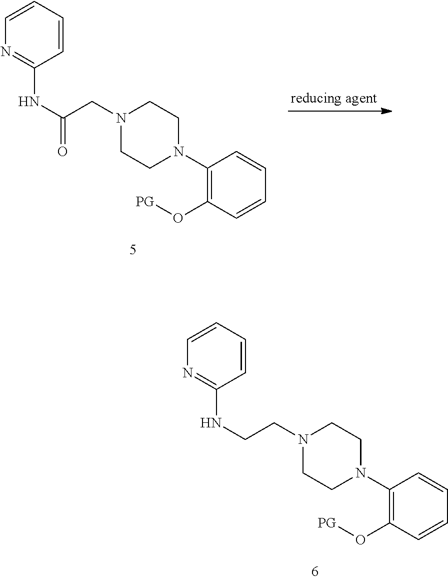

Non-limiting examples of suitable reducing agents for this step include lithium aluminium hydride and lithium borohydride.

Alternatively, intermediate 6 might be arrived at by reduction of the methylated derivative of intermediate 5 (i.e. where methyl is present rather than PG) to remove the amide oxygen resulting in the methylated version of intermediate 6 (i.e where methyl is present rather than PG), and then demethylating this product to obtain intermediate 6 wherein PG is hydrogen. The protecting group PG can be added using known methods if desired. Non-limiting examples of suitable means to carry out the reduction and demethylation steps are as described elsewhere herein.

Intermediate 6 can then be coupled to 4-carbomethoxycyclohexane-1-carboxylic acid 7, yielding 8:

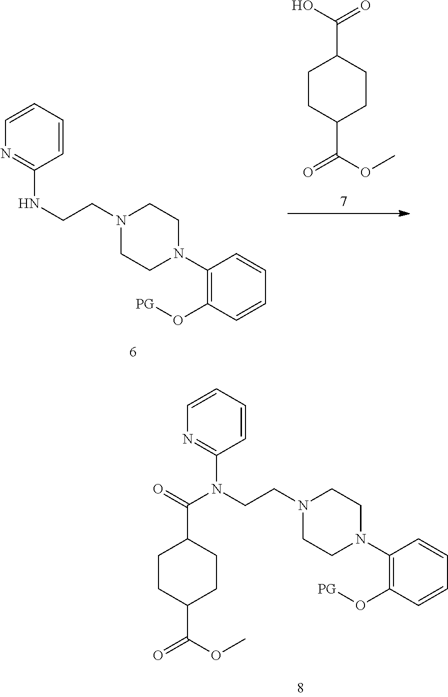

Non-limiting examples of suitable coupling agents include dicyclohexyl carbodiimide, 2-(1H-7-Azabenzotriazol-1-yl)-1,1,3,3-tetramethyl uronium hexafluorophosphate Methanaminium (HATU), benzotriazol-1-yl-oxytripyrrolidinophosphonium hexafluorophosphate (PyBOP), or other benzotriazole-based peptide coupling reagents.

Reduction of the carbomethoxy group of 8 yields intermediate 9. Suitable reducing agents for this step are known to those skilled in the art, and non-limiting examples are described above.

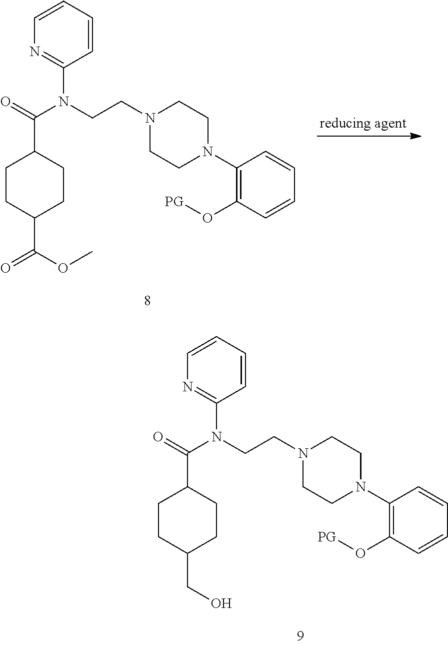

Intermediate 9 can then be converted using known methods, and subsequently deprotected where PG is a protecting group as defined elsewhere herein, to obtain compounds of the present invention, e.g.:

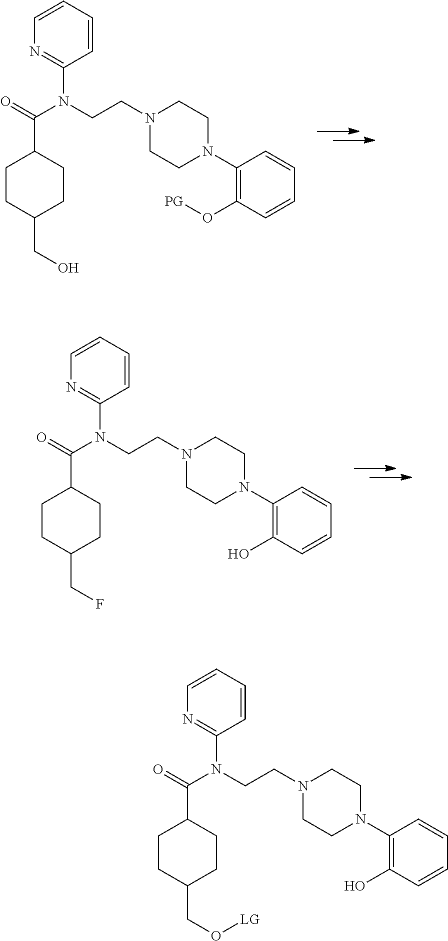

wherein LG is a leaving group. A suitable leaving group in the context of the present invention is a chemical group that can be displaced by nucleophilic displacement reaction with fluoride ion. These are well-known in the art of synthetic chemistry. Non-limiting examples of suitable such leaving groups include: mesylate, tosylate, brosylate, nosylate and the like; and acyloxy groups, such as acetoxy, trifluoroacetoxy, and substituted benzyloxy. Preferred are mesylate, tosylate, brosylate, nosylate and the like, with mesylate and tosylate being more preferred.

An alternative way to obtain a compound of the present invention would be to carry out the synthesis of its methylated derivative according to the methods described by Choi of al (supra), and to then carry out a demethylation step to arrive at compounds falling within the terms of the present invention, e.g.:

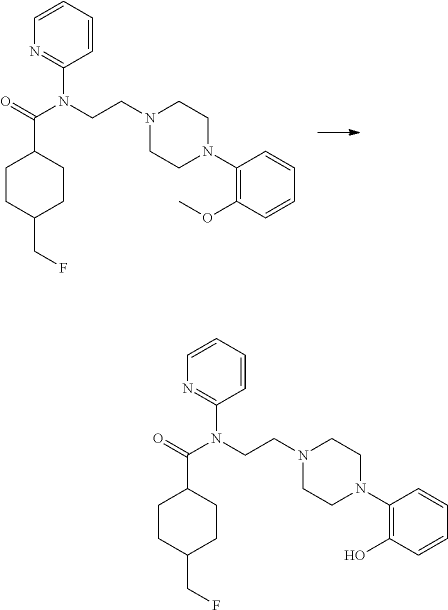 [SMILES: COc1ccccc1N1CCN(CCN(C(=O)C2CCC(CF)CC2)c2ccccn2)CC1 ; O=C(C1CCC(CF)CC1)N(CCN1CCN(c2ccccc2O)CC1)c1ccccn1]

The demethylation step can be carried out as described elsewhere herein. It is noted that BBr₃ is not suitable for this step as it is known to replace the fluoro with bromo.

In a yet further embodiment the present invention provides a method of making the compound of Formula I as defined herein wherein said isotope of fluorine is ¹⁸F, said method comprising:
(i) reacting a precursor compound of Formula II:

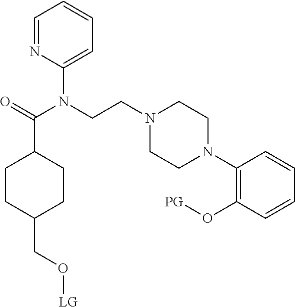

with a source of ¹⁸F fluoride, wherein LG represents hydrogen or a suitable leaving group and PG represents hydrogen or a suitable protecting group; and then,
(ii) removing said protecting group to obtain said compound of Formula I

Suitable and preferred leaving groups and protecting groups are as previously defined herein.

The method to obtain the ¹⁸F-labelled compound of Formula I may additionally comprise:
(i) removal of excess [¹⁸F]fluoride; and/or,
(ii) removal of organic solvent; and/or,
(iii) formulation of the resultant compound together with a biocompatible carrier to obtain a radiopharmaceutical composition suitable for mammalian administration.

In a preferred embodiment, said method of making said compound of Formula I wherein said isotope of fluorine is ¹⁸F is an automated method.

### Kit and Cassette

In another embodiment, the present invention provides a kit for the preparation of the compound of Formula I as defined herein wherein R¹ is ¹⁸F wherein said kit comprises:
(i) a vessel comprising the precursor compound of Formula II as defined herein and
(ii) means for eluting the vessel with a source of ¹⁸F⁻.

The kit may further comprise:
(iii) an ion-exchange cartridge for removal of excess ¹⁸F⁻.

[¹⁸F]fluoride (¹⁸F⁻) for radiofluorination reactions is normally obtained as an aqueous solution from the nuclear reaction ¹⁸O(p,n)¹⁸F and is made reactive by the addition of a cationic counterion and the subsequent removal of water. A suitable cationic counterion for this purpose should possess sufficient solubility within the anhydrous reaction solvent to maintain the solubility of ¹⁸F⁻. Suitable counterions include large but soft metal ions such as rubidium or caesium, potassium complexed with a cryptand such as Kryptofix™, or tetraalkylammonium salts. A preferred suitable source of [¹⁸F]fluoride is selected from [¹⁸F]potassium fluoride and [¹⁸F]caesium fluoride, most preferably [¹⁸F]potassium fluoride wherein Kryptofix™ is used to activate the [¹⁸F]fluoride ion because of its good solubility in anhydrous solvents and enhanced ¹⁸F⁻ reactivity. ¹⁸F⁻ that has been made reactive in this way, reacted with a precursor compound of Formula II, results in an ¹⁸F-labelled compound of Formula I.

Conveniently, all components of the kit are disposable to minimize the possibilities of contamination between runs and may be sterile and quality assured. Preferably, said kit is a cassette suitable for use with an automated synthesis apparatus.

The synthesis of ¹⁸F-labelled compounds, particularly for use as PET tracers, is currently most conveniently carried out by means of an automated synthesis apparatus, e.g. Tracerlab™ and FASTlab™ (both GE Healthcare). FASTlab™ represents the state of the art in automated PET radiotracer synthesis platforms, so that it is desirable in the development of a new PET radiotracer that its synthesis is compatible with FASTlab™. In a preferred embodiment, the method to obtain the ¹⁸F-labelled compound of the invention is automated, preferably via an automated synthesis apparatus. The radiochemistry is performed on the automated synthesis apparatus by fitting a “cassette” to the apparatus. Such a cassette normally includes fluid pathways, a reaction vessel, and ports for receiving reagent vials as well as any solid-phase extraction cartridges used in post-radiosynthetic clean up steps. The reagents, solvents and other consumables required for the automated synthesis may also be included together with a data medium, such as a compact disc carrying software, which allows the automated synthesiser to be operated in a way to meet the end user's requirements for concentration, volumes, time of delivery etc.

The present invention is illustrated by the following non-limiting examples.

### BRIEF DESCRIPTION OF THE EXAMPLES

Example 1 describes the synthesis of (1r,4r)-4-(fluoromethyl)-N-(2-(4-(2-methoxyphenyl)piperazin-1-yl)ethyl)-N-(pyridin-2-yl)cyclohexanecarboxamide (trans-MeFWAY).

Example 2 describes the synthesis of (1r,4r)-4-(fluoromethyl)-N-(2-(4-(2-((2-methoxyethoxy) methoxy)phenyl)piperazin-1-yl)ethyl)-N-(pyridin-2-yl) cyclohexanecarboxamide.

Example 3 describes the synthesis of (1r,4r)-4-([¹⁸F]fluoromethyl)-N-(2-(4-(2-hydroxyphenyl)piperazin-1-yl)ethyl)-N-(pyridin-2-yl)cyclohexanecarboxamide.

Example 4 describes the synthesis of (1s,4s)-4-(fluoromethyl)-N-(2-(4-(2-methoxyphenyl)piperazin-1-yl)ethyl)-N-(pyridin-2-yl)cyclohexanecarboxamide

### LIST OF ABBREVIATIONS USED IN THE EXAMPLES

Boc tert-Butyloxycarbonyl
DAST Diethylaminosulfur trifluoride
DCM Dichloromethane
DMF Dimethyl formamide
LC-MS liquid chromatography-mass spectrometry
MEM 2-Methoxyethoxymethyl
NMR nuclear magnetic resonance
OTs Tosylate
PG protecting group
TEA Triethyl amine
TFA Trifluoroacetic acid

### Example 1

### Synthesis of (1r,4r)-4-(fluoromethyl)-N-(2-(4-(2-methoxyphenyl)piperazin-1-yl)ethyl)-N-(pyridin-2-yl)cyclohexanecarboxamide (MeFWAY)

### 1(i) 2-chloro-N-(pyridin-2-yl)acetamide

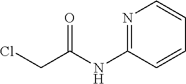 [SMILES: O=C(CCl)Nc1ccccn1]

To a solution of 2-aminopyridine (2 g, 21.3 mmol) and TEA (3.23 g, 31.9 mmol, 4.4 mL) in anhydrous DCM (20 mL) was slowly added chloroacetyl chloride (3.96 g, 35.1 mmol, 2.8 mL) at 0° C. The reaction mixture was stirred at room temperature under a nitrogen atmosphere for 18 h. The reaction mixture was partitioned between DCM (50 mL) and water (50 mL); the organic portion was dried (phase separation cartridge) and evaporated to dryness to afford a brown oil.

The residue was purified by column chromatography on silica gel eluting with petroleum ether (A): ethyl acetate (B) (15-50% (B), 40 g, 10.0 CV, 40 mL/min) to afford a beige solid (2.31 g, 64%). The ¹H NMR indicated presence of both starting materials so the product was re-purified by column chromatography on high performance silica gel eluting with petroleum ether (A): ethyl acetate (B) (40-75% (B), 40 g, 18.3 CV, 40 mL/min) to afford the product as a beige solid (1.92 g, 53%).

LC-MS: m/z calcd for C₇H₇ClN₂O, 170.0. found, 171.0 (M+H)⁺.

¹H NMR (300 MHz, CDCl₃): δ_{H} 4.18 (2H, s, CH₂), 7.06-7.10 (1H, m, pyridyl-5-CH), 7.68-7.75 (1H, m, pyridyl-4-CH), 8.17 (1H, d, J=8.3 Hz, pyridyl-3-CH), 8.30 (1H, dd, J=4.9 Hz and 1.0 Hz, pyridyl-6-CH) and 8.98 (1H, s, NH). ¹³C NMR (75 MHz, CDCl₃): δ_{C} 42.8 (CH₂), 113.9 (pyridyl-3-CH), 120.5 (pyridyl-5-CH), 138.5 (pyridyl-4-CH), 147.9 (pyridyl-6-CH), 150.4 (pyridyl-2-CN) and 164.5 (C═O).

### 1(ii) 2-(4-(2-methoxyphenyl)piperazin-1-yl)-N-(pyridin-2-yl)acetamide

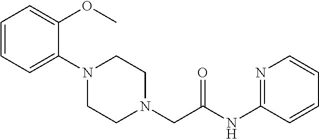 [SMILES: COc1ccccc1N1CCN(CC(=O)Nc2ccccn2)CC1]

To a solution of 1-(2-methoxyphenyl)piperazine (2.16 g, 11.25 mmol) in DMF (20 mL) was added potassium carbonate (3.89 g, 28.14 mmol) and was stirred at 80° C. for one hour. To the cooled reaction mixture was added a solution of 2-chloro-N-(pyridin-2-yl)acetamide (1.92 g, 11.25 mmol) in DMF (10 mL) and sodium iodide (253 mg, 1.69 mmol) and was stirred at 80° C. for 3 h. The cooled reaction mixture was partitioned between ethyl acetate (2*50 mL) and water (50 mL) and the organic portion was dried (MgSO₄), filtered and evaporated to dryness. The residue was purified by column chromatography on silica gel eluting with petroleum ether (A): ethyl acetate (B) (50-100% a (B), 100 g, 27.0 CV, 60 mL/rnin) to afford the desired product as an off-white gum (2.81 g, 77%).

LC-MS: m/z calcd for C₁₈H₂₂N₄O₂ 326.2. found, 327.0.

¹H NMR (300 MHz, CDCl₃): δ_{H} 2.82 (4H, t, J=4.8 Hz, 2′- & 6′-CH₂), 3.17 (4H, br s, 3′- & 5′-CH₂), 3.23 (2H, s, CH₂), 3.86 (3H, s, OCH₃), 6.85-7.06 (5H, m, 4× phenyl-CH and pyridyl-5-CH), 7.70 (1H, td, J 7.8 Hz and 1.9 Hz, pyridyl-4-CH), 8.24-8.32 (2H, m, pyridyl-3-CH and pyridyl-6-CH) and 9.63 (1H, s, NH). ¹³C NMR (75 MHz, CDCl₃): δ_{C} 50.6 (3′- & 5′-CH₂), 53.8 (4′- & 6′-CH₂), 55.3 (OCH₃), 62.2 (CH₂), 111.2 (phenyl-3-CH), 113.8 (pyridyl-3-CH), 118.3 (phenyl-5-CH), 119.8 (phenyl-4-CH), 121.0 (phenyl 6-CH), 123.1 (pyridyl-5-CH), 138.3 (pyridyl-4-CH), 140.9 (phenyl-2-C), 147.9 (pyridyl-6-C), 151.0 (pyridyl-2-C), 152.2 (phenyl-1-C) and 169.2 (C═O).

### 1(iii) N-(2-(4-(2-methoxyphenyl)piperazin-1-yl)ethyl)pyridin-2-amine

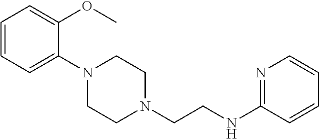 [SMILES: COc1ccccc1N1CCN(CCNc2ccccn2)CC1]

To a solution of 2-(4-(2-methoxyphenyl)piperazin-1-yl)-N-(pyridin-2-yl)acetamide (5.8 g, 17.8 mmol) in THF (80 mL) at 0° C. was slowly added LiAlH₄ (2.02 g, 53.3 mmol, 26.7 mL of a 2.0 M solution in THF) and was stirred at ambient temperature for three hours. The reaction mixture was cooled to 0° C. and quenched with saturated ammonium chloride solution (10 mL); this was then filtered with ethyl acetate and the resultant solution was partitioned between ethyl acetate (150 mL) and water (150 mL). The organic portion was dried (MgSO₄), filtered and evaporated to dryness to afford the desired product as a yellow oil (4.37 g, 79%).

LC-MS: m/z calcd for C₁₈H₂₄N₄O, 312.2. found, 313.1.

¹H NMR (300 MHz, CDCl₃): δ_{H} 2.69 (6H, t, J=6.0 Hz, 2″-CH₂ and 2′- & 6′-CH₂), 3.10 (4H, br s, 3′- & 5′-CH₂), 3.37 (2H, q, J=5.8 Hz, 1″-CH₂), 3.86 (3H, s, OCH₃), 5.13 (1H, br s, NH), 6.41 (1H, d, J=8.6 Hz, pyridyl-5-CH), 6.57 (1H, ddd, J=7.0 Hz, 5.2 Hz and 0.9 Hz, pyridyl-5-CH), 6.84-7.02 (4H, m, 4× phenyl-CH), 7.41 (1H, ddd, J=8.4 Hz, 7.1 Hz and 1.9 Hz, pyridyl-4-CH) and 8.09 (1H, ddd, J=4.9 Hz, 1.8 Hz and 0.9 Hz, pyridyl-6-CH). ¹³C NMR (75 MHz, CDCl₃): δ_{C} 38.5 (1″-CH₂), 50.6 (3′- & 5′-CH₂), 53.1 (4′- & 6′-CH₂), 55.3 (OCH₃); 56.8 (2″-CH₂), 107.0 (pyridyl-3-CH), 111.1 (phenyl-3-CH), 112.6 (pyridyl-5-CH), 118.2 (phenyl-5-CH), 121.0 (phenyl-4-CH), 122.9 (phenyl-6-CH), 137.3 (pyridyl-4-CH), 141.3 (phenyl-2-C), 148.2 (pyridyl-6-CH), 152.2 (pyridyl-2-C) and 158.8 (phenyl-1-C).

### 1(iv) (1r,4r)-4-((2-(4-(2-methoxyphenyl)piperazin-1-yl)ethyl)(pyridin-2-yl)carbamoyl)cyclohexanecarboxylic acid

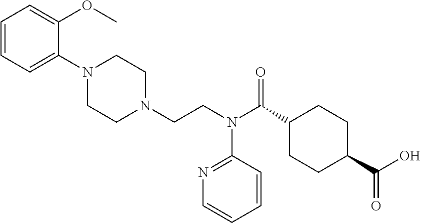

A mixture of trans 1,4-cyclohexanedicarboxlic acid (1 g, 5.813 mmol) and oxalyl chloride (7.4 g, 58.2 mmol, 5 mL) was heated to reflux for 1 h. The excess oxalyl chloride was co-distilled using dichloromethane under nitrogen atmosphere. The solid obtained was dissolved in DCM (50 mL). To the resulting mixture, a solution of N-(2-(4-(2-methoxyphenyl)piperazin-1-yl)ethyl)pyridin-2-amine (1.45 g, 4.65 mmol) and triethylamine (1.152 g, 11.4 mmol, 1.6 mL) in DCM (50 mL) was added slowly at 25° C. under nitrogen atmosphere. After the complete addition, the mixture was stirred at 25° C. for 1 h. The reaction mixture was quenched with water (20 mL) and the DCM layer separated and evaporated to obtain a residue. The residue was dissolved in a sodium hydroxide solution (1 g dissolved in 40 mL water) and the resulting aqueous layer was washed with DCM (25 mL×2). The aqueous layer was adjusted to a pH ˜6.5-6.6 (using conc HCl) and extracted with DCM (25 mL×2). The DCM layer was dried over Na₂SO₄ and evaporated to obtain the desired product as white foam (1.1 g, 52%).

LC-MS: m/z calcd for C₂₆H₃₄N₄O₄, 466.3. found, 466.2

¹H NMR (500 MHz, CDCl₃): δ_{H} 1.03-1.86 (10H, m, 6× cyclohexyl-CHH and CH C(═O)N), 2.67-2.87 (6H, m, 3′- & 5′-CH ₂ and 2″-CH ₂), 3.04 (4H, br s, 4′- & 6′-CH ₂), 3.83 (3H, s, phenyl-OCH ₃), 3.95 (2H, m, 1″-CH ₂), 6.95-7.01 2 (4H, m, 4× phenyl-CH ), 7.20-7.32 (2H, m, pyridyl-3-CH , pyridyl-5-CH ), 7.72-7.78 (1H, t, J=5 Hz, pyridyl-4-CH ), and 8.52 (1H, d, J=5 Hz, pyridyl-6-CH )

### 1(v) (1r,4r)-4-(hydroxymethyl)-N-(2-(4-(2-methoxyphenyl)piperazin-1-yl)ethyl)-N-(pyridin-2-yl)cyclohexanecarboxamide

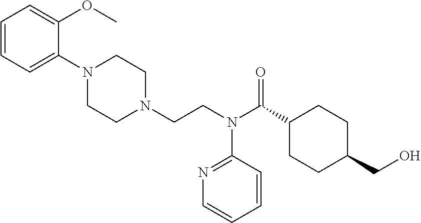

(1r,4r)-4-((2-(4-(2-methoxyphenyl)piperazin-1-yl)ethyl)(pyridin-2-yl)carbamoyl)cyclohexanecarboxylic acid (700 mg, 1.5 mmol) was dissolved in dry THF (15 mL) and cooled to 0° C. Borane-tetrahydrofuran complex (2.0 g, 23.25 mmol, 23.0 mL) was added to the cold solution in three equal lots, every 1 h. After the complete addition, the mixture was stirred at 25° C. for 1 h. The reaction mixture was quenched with water (1 mL) and THF evaporated. The residue obtained was dissolved in methanol (10 mL) and heated to reflux for 1 h. Evaporated methanol and the residue (containing high boiling) was co-distilled using hexane (100 mL×3) to obtain the crude product (0.65 g, 97%), which was used in the next step without further purification.

LC-MS: m/z calcd for C26H36N403, 452.3. found, 452.3

### 1(vi) ((1r,4r)-4-((2-(4-(2-methoxyphenyl)piperazin-1-yl)ethyl)(pyridin-2-yl)carbamoyl)cyclohexyl)methyl 4-methylbenzenesulfonate

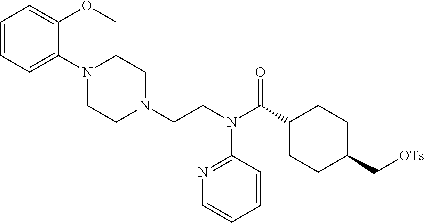

To a solution of (1r,4r)-4-(hydroxymethyl)-N-(2-(4-(2-methoxyphenyl)piperazin-1-yl)ethyl)-N-(pyridin-2-yl)cyclohexanecarboxamide (850 mg, 1.88 mmol) in DCM (10 mL) was added tosyl chloride (1 g, 5.2 mmol) and TEA (0.72 g, 7.12 mmol, 1 mL). The mixture was stirred at 25° C. for 24 h. The reaction mixture was quenched with 10% aqueous sodium bicarbonate solution (50 mL) and the DCM layer separated. The DCM layer was dried (Na₂SO₄) and evaporated to dryness. The residue was purified by manual column chromatography on neutral alumina (100 g) eluting with Hexane (A): Ethyl acetate (B) (10-50% (B), to afford the desired product as foam on drying under high vacuum (550 mg, 48%).

LC-MS: m/z calcd for C33H42N4O5S, 606.3. found, 605.6

1H NMR (300 MHz, CD₃CN): δH 0.71 (2H, q, J=12 Hz, 2× cyclohexyl-CHH), 1.34-1.83 (7H, in, 6× cyclohexyl-CHH and CHC(═O)N), 1.96 (1H, t, J=10.5 Hz, cyclohexyl-CHCH₂OTs), 2.44 (3H, s, tosyl-CH₃), 2.46-2.58 (6H, m, 3′- & 5′-CH₂ and 2″-CH₂), 2.90 (4H, br s, 4′- & 6′-CH₂), 3.75 (2H, d, J=6 Hz, CH₂OTs), 3.79 (3H, s, phenyl-OCH₃), 3.88 (2H, t, J=6.0 Hz, 1″-CH₂), 6.82-7.04 (4H, m, 4× phenyl-CH), 7.25-7.48 (4H, m, pyridyl-3-CH, pyridyl-5-CH and 2× tosyl-CHCCH₃), 7.68-7.88 (3H, m, pyridyl-4-CH and 2× tosyl-CHCSO₂) and 8.48 (1H, d, J=5 Hz, pyridyl-6-CH).

1(vii) (1r,4r)-4-(fluoromethyl)-N-(2-(4-(2-methoxyphenyl)piperazin-1-yl)ethyl)-N-(pyridin-2-yl)cyclohexanecarboxamide (trans-MeFWAY)

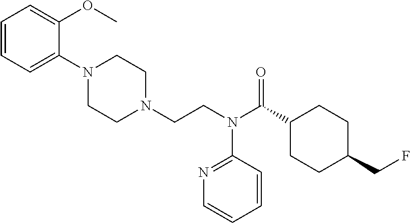

To a solution of (1r,4r)-4-(hydroxymethyl)-N-(2-(4-(2-methoxyphenyl)piperazin-1-yl)ethyl)-N-(pyridin-2-yl)cyclohexanecarboxamide (40 mg, 0.09 mmol) in DCM (2 mL) in an ice-water bath was added DAST (21 mg, 0.13 mmol, 17 uL) and was stirred at ambient temperature under a nitrogen atmosphere for 94 h. The reaction mixture was quenched with 10% aqueous sodium bicarbonate solution (10 mL) and partitioned between the aqueous and DCM (10 mL). The organic portion was dried (phase separation cartridge) and evaporated to dryness. The residue was purified by column chromatography on silica gel eluting with DCM (A): methanol (B) (2-10% (B), 4 g, 76.0 CV, 18 mL/min) to afford the desired product as a colourless oil (14 mg, 35%).

This compound can be demethylated using methods described hereinabove to result in a compound of the present invention.

LC-MS: m/z calcd for C₂₆H₃₅FN₄O₂, 454.3. found 455.2 (M+H)⁺.

¹H NMR (300 MHz, CDCl₃): δ_{H} 0.83 (2H, q, J 11.7 Hz, 2× cyclohexyl-CHH), 1.54-1.86 (7H, m, 6× cyclohexyl-CHH and cyclohexyl-CHC(═O)N), 2.19 (1H, t, J=11.9 Hz, cyclohexyl-CHCH₂F), 2.61 (6H, m, 2× piperazinyl-CH₂ and 2″-CH₂), 2.98 (4H, br s, 2× piperazinyl-CH₂), 3.84 (3H, s, phenyl-OCH₃), 3.98 (2H, t, J=6.9 Hz, 1″-CH₂), 4.15 (2H, dd, J_{CF}=47.7 Hz, J=5.4 Hz, CH₂F), 6.83-7.01 (4H, m, 4× phenyl-CH), 7.22-7.31 (2H, m, pyridyl-3-CH and pyridyl-5-CH), 7.76 (1H, td, J=7.7 Hz and 1.8 Hz, pyridyl-4-CH) and 8.52 (1H, dd, J=4.9 Hz and 1.2 Hz, pyridyl-6-CH). ¹³C NMR (75 MHz, CDCl₃): δ_{C} 27.4 (2× cyclohexyl-CH₂(CHCH₂F)), 28.7 (2× cyclohexyl-CH₂(CHC(═O)N)), 37.7 (cyclohexyl-CH(CH₂F), 42.1 (cyclohexyl-CHC(═O)N), 45.3 (1″-CH₂), 50.6 (2′- & 6′-CH₂), 53.4 (2″-, 3′- & 5′-CH₂), 55.3 (phenyl-OCH₃), 111.2 (phenyl-3-CH), 118.1 (phenyl-5-CH), 120.9 (phenyl-4-CH), 122.2 (phenyl-6-CH), 122.8 (pyridyl-5-CH and pyridyl-3-CH), 138.2 (pyridyl-4-CH), 142.3 (phenyl-2-CO), 149.3 (pyridyl-6-CH), 152.2 (phenyl-1-CN) and 175.8 (C═O). ¹⁹F NMR (283 MHz, CDCl3): δ_{F} −223.9.

### Example 2

### Synthesis of (1r,4r)-4-(fluoromethyl)-N-(2-(4-(2-((2-methoxyethoxy) methoxy)phenyl)piperazin-1-yl)ethyl)-N-(pyridin-2-yl)cyclohexanecarboxamide

### 2(i) tert-butyl 4-(2-hydroxyphenyl)piperazine-1-carboxylate

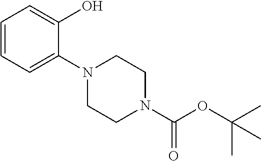 [SMILES: CC(C)(C)OC(=O)N1CCN(c2ccccc2O)CC1]

To a solution of 2-(1-piperazino)phenol (3.0 g, 16.8 mmol) and NaHCO₃ (2.12 g, 25.3 mmol) in a 1:1:1 mixture of THF/H₂O/dioxane (60 mL) was added Boc₂O (4.41 g, 20.2 mmol) and was stirred at ambient temperature for 20 mins until a solid formed. The reaction mixture was filtered and the filtrate was partitioned between water (100 mL) and DCM (100 mL); the organic portion was dried (phase separation cartridge) and evaporated to dryness. The combined residue and solid product were recrystallized from boiling petroleum ether to afford tert-butyl 4-(2-hydroxyphenyl)piperazine-1-carboxylate as a beige solid (3.38 g, 72%).

LC-MS: m/z calcd for C₁₅H₂₂N₂O₃, 278.2. found, 277.0 (M−H)⁺.

¹H NMR (301 MHz, CHLOROFORM-D) δ 7.14-7.05 (m, 2H, phenyl-3-CH and phenyl-4-CH), 6.98-6.93 (m, 1H, 6-CH), 6.89-6.83 (m, 1H, 5-CH), 3.63-3.53 (m, 4H, 2′- & 6′-CH₂), 2.87-2.77 (m, 4H, 3′- & 5′-CH₂), 1.50-1.48 (s, 9H, 3×CH₃).

### 2(ii) tert-butyl 4-(2-((2-methoxyethoxy)methoxy)phenyl)piperazine-1-carboxylate

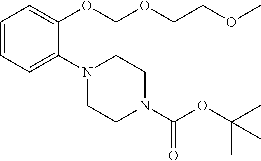 [SMILES: COCCOCOc1ccccc1N1CCN(C(=O)OC(C)(C)C)CC1]

To a solution of tert-butyl 4-(2-hydroxyphenyl)piperazine-1-carboxylate (3.30 g, 11.9 mmol) in DMF (100 mL) at 0° C. was slowly added sodium hydride (474 mg of a 60% dispersion in mineral oil, 11.9 mmol) and was stirred for 30 mins. Thereto was then added MEM-Chloride (1.48 g, 11.9 mmol, 1.35 mL) and was stirred at 60° C. for 18 h. The reaction mixture was evaporated to dryness and the residue was partitioned between ethyl acetate (2*75 mL) and water (75 mL). The organic portion was washed with brine (75 mL), dried over magnesium sulfate, filtered and evaporated to dryness. The residue was purified by column chromatography on silica gel eluting with petroleum ether (A): ethyl acetate (B) (10-40% (B), 50 g, 20.0 CV, 40 mL/min) to afford tert-butyl 4-(2-((2-methoxyethoxy)methoxy)phenyl)piperazine-1-carboxylate as a colourless oil (937 mg, 22%).

¹H NMR (301 MHz, CHLOROFORM-D) δ 7.15-7.09 (m, 1H, —CH), 7.01-6.88 (m, 3H, phenyl-4-CH, phenyl-5-CH and phenyl-6-CH), 5.33-5.29 (s, 2H, OCH₂O), 3.89-3.83 (m, 2H, CH₃OCH₂), 3.60-3.54 (m, 6H, and 2′- & 6′-CH₂), 3.39-3.36 (m, 3H, OCH₃), 3.03-2.96 (t, J=5.0 Hz, 4H, 3′- & 5′-CH₂), 1.49-1.45 (s, 9H, 3×CH₃).

### 2(iii) 1-(2-((2-methoxyethoxy)methoxy)phenyl)piperazine (4, PG=MEM)

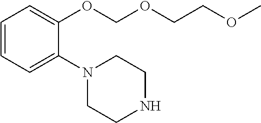 [SMILES: COCCOCOc1ccccc1N1CCNCC1]

tert-Butyl 4-(2-((2-methoxyethoxy)methoxy)phenyl)piperazine-1-carboxylate (900 mg, 2.46 mmol) was slowly dissolved in neat TFA (5 mL) and was stirred at ambient temperature for 10 mins. The reaction mixture was diluted with ether (50 mL) and neutralised with saturated potassium carbonate solution (10 mL) at 0° C. The aqueous layer was washed with diethyl ether (2*50 mL) and the combined organics were dried over magnesium sulfate, filtered and evaporated to dryness to afford a pale yellow residue. The aqueous layer was then basified with additional saturated potassium carbonate solution (5 mL) and the residue was re-dissolved in DCM (10 mL) and partitioned with water and additional DCM (2*30 mL) The organic portion was dried (phase separation cartridge) and evaporated to dryness to afford 1-(2-((2-methoxyethoxy)methoxy)phenyl)piperazine as a pale yellow oil (450 mg, 69%).

¹H NMR (301 MHz, CHLOROFORM-D) δ 7.10-7.03 (m, 1H, phenyl-3-CH), 6.96-6.84 (m, 3H, phenyl-4-CH, phenyl-5-CH and phenyl-6-CH), 5.29-5.23 (s, 2H, OCH₂O), 3.90-3.73 (m, 2H, CH₃OCH₂), 3.60-3.43 (m, 2H, CH₂CH₂OCH₂), 3.40-3.25 (s, 3H, OCH₃), 3.11-2.89 (s, 8H, 4× piperazinyl-NCH₂).

### 2(iv): 2-(4-(2-((2-methoxyethoxy)methoxy)phenyl)piperazin-1-yl)-N-(pyridin-2-yl)acetamide (5, PG=MEM)

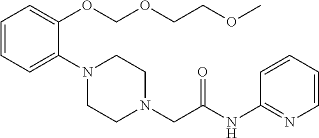 [SMILES: COCCOCOc1ccccc1N1CCN(CC(=O)Nc2ccccn2)CC1]

To a solution of 1-(2-((2-methoxyethoxy)methoxy)phenyl)piperazine (450 mg, 1.69 mmol) in DMF (15 mL) was added potassium carbonate (584 mg, 4.22 mmol) and the mixture stirred at 80° C. for 45 minutes. To the cooled reaction mixture was added 2-chloro-N-(pyridin-2-yl)acetamide 3 (288 mg, 1.69 mmol) and sodium iodide (38 mg, 0.25 mmol) and stirring continued at 80° C. for 3 h. The cooled reaction mixture was evaporated to remove the majority of the DMF and the residue was partitioned between ethyl acetate (50 mL) and water (50 mL). The organic portion was washed with brine (50 mL), dried over magnesium sulfate, filtered and evaporated to dryness and the residue was purified by column chromatography on silica gel eluting with petroleum ether (A): ethyl acetate (B) (40-90% (B), 50 g, 25.0 CV, 40 mL/min) to afford 2-(4-(2-((2-methoxyethoxy)methoxy)phenyl)piperazin-1-yl)-N-(pyridin-2-yl)acetamide as a pale yellow oil (515 mg, 76%).

¹H NMR (301 MHz, CHLOROFORM-D) δ 9.62-9.56 (s, 1H, NH), 8.29-8.25 (ddd, J=4.9, 2.0, 0.9 Hz, 1H, pyridyl-6-CH), 8.25-8.20 (m, 1H, pyridyl-3-CH), 7.70-7.63 (m, 1H, pyridyl-4-CH), 7.11-6.88 (m, 5H, 4× phenyl-CH and pyridyl-5-CH), 5.29-5.26 (s, 2H, OCH₂O), 3.85-3.79 (m, 2H, CH₃OCH₂), 3.56-3.50 (m, 2H, CH₂CH₂OCH₂), 3.35-3.32 (s, 3H, OCH₃), 3.20-3.11 (m, 6H, 2″-CH₂ and 3′- & 5′-CH₂), 2.81-2.71 (=t, J 4.8 Hz, 4H, 2′- & 6′-CH₂). ¹³C NMR (76 MHz, CHLOROFORM-D) δ 169.18 (C═O), 151.08 (phenyl-1-C), 150.10 (pyridyl-2-C), 148.08 (pyridyl-6-CH), 142.14 (phenyl-2-C), 138.39 (pyridyl-4-CH), 123.23 (pyridyl-5-CH), 122.88 (phenyl-6-CH), 119.94 (phenyl-4-CH), 118.82 (phenyl-5-CH), 116.87 (phenyl-3-CH), 113.92 (pyridyl-3-CH), 94.33 (OCH₂O), 71.68 (CH₃OCH₂), 67.99 (CH₂CH₂OCH₂), 62.36 (2″-CH₂), 59.11 (OCH₃), 53.99 (3′- & 5′-CH₂), 50.75 (2′- & 6′-CH₂).

### 2(v): N-(2-(4-(2-((2-methoxyethoxy)methoxy)phenyl)piperazin-1-yl)ethyl)pyridin-2-amine (6, PG=MEM)

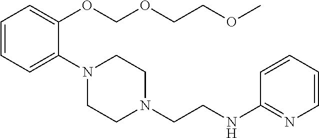 [SMILES: COCCOCOc1ccccc1N1CCN(CCNc2ccccn2)CC1]

To a solution of 2-(4-(2-((2-methoxyethoxy)methoxy)phenyl)piperazin-1-yl)-N-(pyridin-2-yl)acetamide (500 mg, 1.25 mmol) in THF (15 mL) at 0° C. was slowly added LiAlH₄ (142 mg, 3.75 mmol, 1.87 mL of a 2.0 M solution in THF) and was stirred at ambient temperature for three hours. The reaction mixture was cooled to 0° C. and quenched with saturated ammonium chloride solution (3 mL) then filtered with ethyl acetate and the resultant solution was partitioned between ethyl acetate (25 mL) and water (25 mL) The organic portion was dried over magnesium sulfate, filtered and evaporated to dryness to afford a yellow oily residue. The residue was purified by column chromatography on silica gel eluting with dichloromethane (A): methanol (B) (2-10% (B), 50 g, 21.2 CV, 40 mL/min) to afford N-(2-(4-(2-((2-methoxyethoxy)methoxy)phenyl)piperazin-1-yl)ethyl)pyridin-2-amine as a yellow oil (195 mg, 40%).

¹H NMR (301 MHz, CHLOROFORM-D) δ 8.16-8.01 (ddd, J=5.1, 1.9, 0.9 Hz, 1H, pyridyl-6-CH), 7.43-7.36 (m, 1H, pyridyl-4-CH), 7.13-7.08 (m, 1H, phenyl-3-CH), 7.01-6.91 (m, 3H, 4-,5- & 6-phenyl-CH), 6.58-6.52 (ddd, J=7.1, 5.1, 0.9 Hz, 1H, pyridyl-3-CH), 6.43-6.38 (dt, J=8.4, 0.9 Hz, 1H, pyridyl-5-CH), 5.35-5.23 (s, 2H, OCH₂O), 5.18-5.08 (t, J=4.6 Hz, 1H, NH), 3.88-3.82 (m, 2H, CH₃OCH₂), 3.59-3.54 (m, 2H, CH₂CH₂OCH₂), 3.38-3.36 (s, 5H, OCH₃ and 1″-CH₂), 3.12-3.07 (m, 4H, 3′- &5′-CH₂), 2.72-2.62 (m, 6H, 2″-CH₂ and 2′- & 6′-CH₂). ¹³C NMR (76 MHz, CHLOROFORM-D) δ 158.90 (phenyl-1-C), 150.09 (pyridyl-2-C), 148.26 (pyridyl-6-CH), 142.48 (phenyl-2-C), 137.39 (pyridyl-4-CH), 123.00 (phenyl-6-CH), 122.88 (phenyl-4-CH), 118.70 (phenyl-5-CH), 116.89 (phenyl-3-CH), 112.78 (pyridyl-5-CH), 107.15 (pyridyl-3-CH), 94.35 (OCH₂O), 71.71 (CH₃OCH₂), 67.98 (CH₂CH₂OCH₂), 59.13 (OCH₃), 56.89 (2″-CH₂), 53.33 (3′- & 5′-CH₂), 50.74 (2′- & 6′-CH₂), 38.61 (1″-CH₂).

### 2(vi); (1r,4r)-4-((2-(4-(2-((2-methoxyethoxy)methoxy)phenyl)piperazin-1-yl)ethyl)(pyridin-2-yl)carbamoyl)cyclohexanecarboxylic acid (10, PG=MEM)

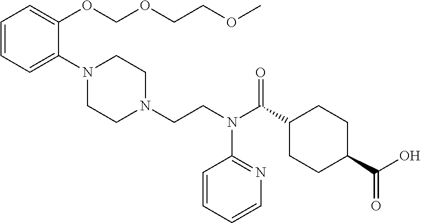

A mixture of trans-1,4-cyclohexanedicarboxlic acid (1 g, 5.813 mmol) and oxalyl chloride (7.4 g, 58.2 mmol, 5 mL) was heated to reflux for 1 h. The excess oxalyl chloride was co-distilled using dichloromethane under nitrogen atmosphere. To a solution of a portion of the 1,4-cyclohexane diacid chloride (120 mg, 0.57 mmol) in DCM (5 mL) was added a solution of N-(2-(4-(2-((2-methoxyethoxy)methoxy)phenyl)piperazin-1-yl)ethyl)pyridin-2-amine (178 mg, 0.46 mmol) and TEA (64 mg, 0.63 mmol, 0.09 mL) in DCM (5 mL) and was stirred at ambient temperature for 1 hour.

The reaction mixture was quenched with water (4 mL) and the organic portion was evaporated to dryness The residue was dissolved in 10% sodium hydroxide solution (1 mL), diluted with water (10 mL) and DCM (10 mL). The organic portion was collected and the aqueous was adjusted to pH 6.5 using conc. HCl and extracted with DCM (2*30 mL) and the combined organic portions were dried (phase sep cartridge) and evaporated to dryness to afford 13 mg of a colourless oil. To the aqueous portion was added diethyl ether (50 mL); the organic portion was dried over magnesium sulfate, filtered, combined with the colourless oil and evaporated to dryness to afford (1s,4s)-4-((2-(4-(2-((2-methoxyethoxy) methoxy)phenyl)piperazin-1-yl)ethyl)(pyridin-2-yl)carbamoyl)cyclohexanecarboxylic acid (240 mg, 77%) in total.

¹H NMR (301 MHz, CHLOROFORM-D) δ 8.57-8.42 (m, 1H, pyridyl-6-CH), 7.82-7.68 (m, 1H, pyridyl-4-CH), 7.32-7.17 (m, 2H, pyridyl-3-CH and pyridyl-5-CH), 7.15-7.03 (m, 1H, phenyl-3-CH), 7.02-6.81 (m, 3H, 3× phenyl-CH), 5.39-5.14 (m, 2H, OCH₂O), 4.05-3.72 (m, 2H, 1″-CH₂), 3.72-3.22 (m, 7H, 2×OCH₂ and OCH₃), 3.02-2.95 (s, 4H, 2× piperazinyl-CH₂), 2.75-2.52 (m, 6H, 2× piperazinyl-CH₂ and 2″-CH₂), 2.34-2.09 (m, 2H, 2× cyclohexyl-CH), 2.07-1.68 (m, 4H, 4× cyclohexyl-CHH), 1.68-1.53 (m, 2H, 2× cyclohexyl-CHH), 1.36-1.08 (m, 2H, 2× cyclohexyl)

### 2(vii) (1r,4r)-4-(hydroxymethyl)-N-(2-(4-(2-((2-methoxyethoxy) methoxy)phenyl)piperazin-1-yl)ethyl)-N-(pyridin-2-yl)cyclohexanecarboxamide (12, PG=MEM)

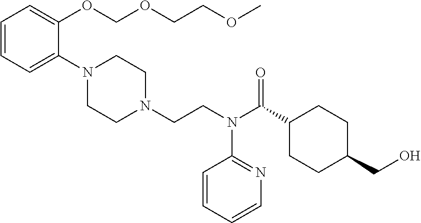

To a solution of (1r,4r)-4-((2-(4-(2-((2-methoxyethoxy)methoxy)phenyl)piperazin-1-yl)ethyl)(pyridin-2-yl)carbamoyl)cyclohexanecarboxylic acid (240 mg, 0.44 mmol) in anhydrous THF (4 mL) at 0° C. was added borane-THF complex (191 mg, 2.22 mmol, 2.22 mL of a 1.0 M solution in THF) once an hour for three hours. After complete addition, the reaction mixture was stirred at ambient temperature for one hour. The reaction mixture was quenched with water (2 mL) and evaporated. The residue was dissolved in methanol (10 mL) and heated at reflux for one hour. The reaction mixture was evaporated to dryness to afford a colourless solid residue (520 mg) that was insoluble in chloroform and sparingly soluble in methanol. ¹H NMR indicated the presence of a large amount of water so the residue was partitioned between water (20 mL) and diethyl ether (50 mL). The organic portion was dried over magnesium sulfate, filtered and evaporated to dryness. The residue was purified by column chromatography on high performance silica gel eluting with DCM (A): methanol (B) (2-10% (B), 12 g, 28.0 CV, 30 mL/min) to afford (1r,4r)-4-(hydroxymethyl)-N-(2-(4-(2-((2-methoxyethoxy)methoxy)phenyl)piperazin-1-yl)ethyl)-N-(pyridin-2-yl)cyclohexanecarboxamide as a colourless oil (65 mg, 28%).

LC-MS: m/z calcd for C₂₆H₃₆N₄O₃, 526.3. found, 527.3 (M+H)⁺.

¹H NMR (301 MHz, CHLOROFORM-D) δ 8.56-8.43 (m, 1H, pyridyl-6-CH), 7.82-7.68 (m, 1H, pyridyl-4-CH), 7.32-7.18 (m, 2H, pyridyl-3-CH and pyridyl-5-CH), 7.00-6.80 (m, 4H, 4× phenyl-CH), 5.28-5.24 (d, J=2.6 Hz, 2H, OCH₂O), 3.87-3.79 (m, 2H, CH₃OCH₂), 3.59-3.52 (m, 2H, CH₂CH₂OCH₂), 3.38-3.35 (m, 5H, OCH₃ and 1″-CH₂), 3.00-2.93 (s, 4H, 3′- &5′-CH₂), 2.63-2.49 (m, 6H, 2″-CH₂ and 2′- & 6′-CH₂), 1.88-1.70 (m, 4H, 4× cyclohexyl-CHH), 1.70-1.21 (m, 4H, 4× cyclohexyl-CHH), 1.06-0.83 (m, 1H, cyclohexyl-CH), 0.83-0.64 (m, 1H, cyclohexyl-CH).

¹³C NMR (76 MHz, CHLOROFORM-D) δ 176.03 (C═O), 150.03 (pyridyl-6-CH), 149.22 (pyridyl-2-C), 142.37 (phenyl-1-C), 138.29 (phenyl-2-C), 138.12 (pyridyl-4-CH), 122.89 (pyridyl-3-CH), 122.81 (pyridyl-5-CH), 122.32 (phenyl-6-CH), 118.55 (phenyl-4-CH), 116.87 (phenyl-5-CH), 111.18 (phenyl-3-CH), 94.31 (OCH₂O), 71.69 (CH₃OCH₂), 68.34 (CH₂CH₂OCH₂), 67.95 (CH₂OH), 59.14 (OCH₃), 53.55 (3′- & 5′-CH₂), 50.70 (2′- & 6′-CH₂), 42.47 (cyclohexyl-CHC(═O)N), 39.70 (cyclohexyl-CH(CH₂OH), 33.63 (1″-CH₂), 29.01 (2× cyclohexyl-CH₂(CHC(═O)N)), 28.57 (2× cyclohexyl-CH₂(CHCH₂OH)).

### 2(viii) (1r,4r)-4-(fluoromethyl)-N-(2-(4-(2-((2-methoxyethoxy) methoxy)phenyl)piperazin-1-yl)ethyl)-N-(pyridin-2-yl)cyclohexanecarboxamide

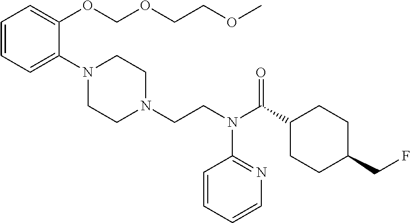

To a solution of (1r,4r)-4-(hydroxymethyl)-N-(2-(4-(2-((2-methoxyethoxy)methoxy) phenyl)piperazin-1-yl)ethyl)-N-(pyridin-2-yl)cyclohexanecarboxamide (65 mg, 0.12 mmol) in DCM (5 mL) in an ice-water bath was added DAST (40 mg, 0.25 mmol, 32 uL) and the solution was stirred at ambient temperature for 23 hours. The reaction mixture was quenched with 10% aqueous sodium bicarbonate solution (10 mL) and partitioned between the aqueous and DCM (20 mL). The organic portion was dried (phase separation cartridge) and evaporated to dryness. The residue was purified by column chromatography on high performance silica gel eluting with DCM (A): methanol (B) (2-10% (B), 12 g, 28.0 CV, 30 mL/min) to afford (1r,4r)-4-(fluoromethyl)-N-(2-(4-(2-((2-methoxyethoxy)methoxy)phenyl)piperazin-1-yl)ethyl)-N-(pyridin-2-yl)cyclohexanecarboxamide as a colourless solid (7 mg).

### 2(ix) 1r,4r)-4-(fluoromethyl)-N-(2-(4-(2-hydroxyphenyl)piperazin-1-yl)ethyl)-N-(pyridin-2-yl)cyclohexanecarboxamide

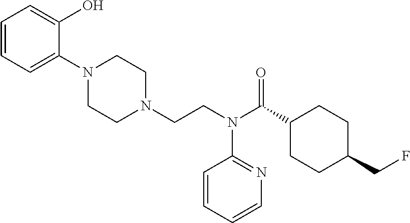

To a solution of (1r,4r)-4-(fluoromethyl)-N-(2-(4-(2-((2-methoxyethoxy)methoxy) phenyl)piperazin-1-yl)ethyl)-N-(pyridin-2-yl)cyclohexanecarboxamide (7 mg, 13.2 umol) in DCM (1 mL) was added TFA (0.5 mL) and the solution stirred at ambient temperature for 4 days. The reaction mixture was quenched with saturated potassium carbonate solution and partitioned between DCM (10 mL) and water (10 mL); the organic portion was dried (phase separation cartridge) and evaporated to dryness. The residue was purified by column chromatography on silica gel eluting with DCM (A): methanol (B) (3% (B), 4 g, 30.0 CV, 18 mL/min) to afford (1r,4r)-4-(fluoromethyl)-N-(2-(4-(2-hydroxyphenyl)piperazin-1-yl)ethyl)-N-(pyridin-2-yl)cyclohexanecarboxamide (2 mg)

LC-MS: m/z calcd for C₂₅H₃₃FN₄O₂, 440.3. found, 441.3 (M+H)⁺.

### Example 3

### Synthesis of (1r,4r)-4-([¹⁸F]fluoromethyl)-N-(2-(4-(2-hydroxyphenyl)piperazin-1-yl)ethyl)-N-(pyridin-2-yl)cyclohexanecarboxamide

### 3(i) ((1r,4r)-4-((2-(4-(2-((2-methoxyethoxy)methoxy)phenyl)piperazin-1-yl)ethyl)(pyridin-2-yl)carbamoyl)cyclohexyl)methyl 4-methylbenzenesulfonate

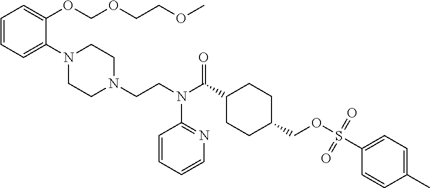

To a solution of (1r,4r)-4-(fluoromethyl)-N-(2-(4-(2-((2-methoxyethoxy)methoxy) phenyl)piperazin-1-yl)ethyl)-N-(pyridin-2-yl)cyclohexanecarboxamide (100 mg, 0.19 mmol) in DCM (5 L) is added tosyl chloride (59 mg, 0.28 mmol) and TEA (5 drops). The mixture is stirred at 25° C. for 24 h. The reaction mixture is quenched with 10% aqueous sodium bicarbonate solution (5 mL) and the DCM layer separated, dried over sodium sulfate and evaporated to dryness. The residue is purified by column chromatography on neutral alumina (100 g) and eluting with hexane (A): ethyl acetate (B) (10-50% (B), to afford ((1r,4r)-4-((2-(4-(2-((2-methoxyethoxy)methoxy) phenyl)piperazin-1-yl)ethyl)(pyridin-2-yl)carbamoyl)cyclohexyl)methyl 4-methylbenzenesulfonate. Deprotection to remove the protecting group on the hydroxyl may be carried out by acid hydrolysis either before or after the radiolabelling step 3(ii).

### 3(ii) (1r,4r)-4-([¹⁸F]fluoromethyl)-N-(2-(4-(2-hydroxyphenyl)piperazin-1-yl)ethyl)-N-(pyridin-2-yl)cyclohexanecarboxamide

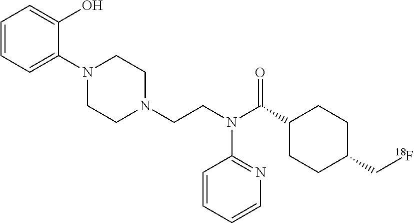

Potassium carbonate solution (50 μL, 0.1 M) is added to Kryptofix™ (5.0 mg) and anhydrous acetonitrile (0.50 mL) in a 3 mL Wheaton vial equipped with a stirrer vane. [¹⁸F]fluoride (aq.) is added to the vial, and heated to 110° C. under a stream of N₂ to azeotropically dry the [¹⁸F]fluoride. Two further portions of anhydrous acetonitrile (2×0.5 mL) are added and similarly dried. The reaction vial is cooled to room temperature, and the precursor ((1r,4r)-4-((2-(4-(2-hydroxyphenyl)piperazin-1-yl)ethyl)(pyridin-2-yl)carbamoyl)cyclohexyl)methyl 4-methylbenzenesulfonate (1.0 mg) in anhydrous DMF (150 μL) is added. The reaction is stirred at 110° C. for 30 mm. The reaction is diluted with acetonitrile (0.6 mL) and water (1.0 mL) and loaded to a semi-preparative HPLC system. The product is collected using a manual switch, diluted with water to a total volume of 20 mL, and loaded onto a tC18 Light Sep-pak cartridge (primed with 1 mL ethanol and 2 mL water). The product is eluted with ethanol (0.5 mL) and diluted with phosphate buffered saline (4.5 mL).

### Example 4

### Synthesis of (1s,4s)-4-(fluoromethyl)-N-(2-(4-(2-methoxyphenyl)-piperazin-1-yl)ethyl)-N-(pyridin-2-yl)cyclohexanecarboxamide

### 4(i) (1s,4s)-4-((2-(4-(2-methoxyphenyl)piperazin-1-yl)ethyl)(pyridin-2-yl)carbamoyl)cyclohexanecarboxylic acid

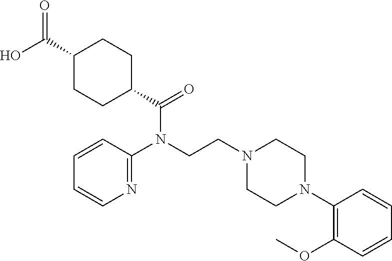 [SMILES: COc1ccccc1N1CCN(CCN(c2ccccn2)C(=O)[C@H]2CC[C@@H](C(=O)O)CC2)CC1]

A mixture of N-(2-(4-(2-methoxyphenyl)piperazin-1-yl)ethyl)pyridin-2-amine (0.9 g, 2.88 mmol) and triethylamine (0.58 g, 5.81 mmol, 0.81 ml) dissolved in DCM (15 ml) and was slowly added to (1s,4s)-cyclohexane-1,4-dicarbonyl dichloride in DCM at 0° C. for 1 h under a dry nitrogen atmosphere. The reaction mixture was stirred for 2 h at room temperature before it was cooled to 0° C. and acidified to pH 2, using concentrated HCl. The DCM layer was separated out. The aqueous layer was then neutralized with solid sodium bicarbonate and the product that precipitated out was extracted into DCM. The DCM layer was dried over anhydrous sodium sulfate and evaporated to obtain crude (1 s,4s)-4-((2-(4-(2-methoxyphenyl)piperazin-1-yl)ethyl)(pyridin-2-yl)carbamoyl)cyclohexanecarboxylic acid (1.4 g). The product was used directly in the next step with no further purification.

LC-MS: m/z calcd for C₂₆H₃₄N₄O₄, 466.3. found, 466.2 (M)⁺.

Reduction and fluorination were carried out under the same conditions as described in Example 1 for the trans-isomer. Demethylation of this compound using any of the methods described hereinabove results in a compound of the present invention.

What is claimed is:

1) A compound of Formula I: 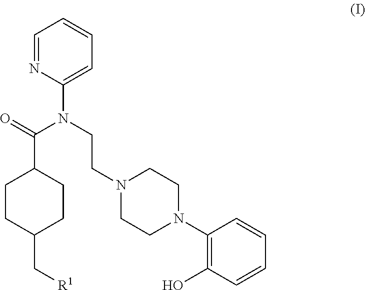
wherein R¹ is an isotope of fluorine.

2) The compound according to claim 1, wherein the compound is a compound of Formula Ia: 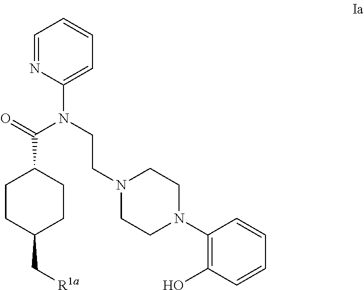
wherein R^{1a} is an isotope of fluorine.

3) The compound according to claim 1, wherein the compound is a compound of Formula Ib: 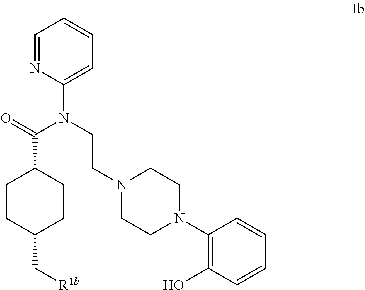
wherein R^{1b} is an isotope of fluorine.

4) A composition comprising the compound according to claim 1 and a physiologically acceptable carrier or vehicle.

5) A method comprising:
(a) administering to a subject in vivo an imaging-effective amount of a compound according to claim 1 wherein said isotope of fluorine is ¹⁸F; and then,
(b) detecting the radioactive emission of said ¹⁸F.

6) The method according to claim 5, wherein said radioactive emission is detected using PET.

7) The method according to claim 5, wherein said radioactive emission is detected in the brain of said subject.

8) The method according to claim 5, wherein said subject is known or suspected to have a neurological disorder.

9) The method according to claim 8, wherein said neurological disorder is an affective disorder, an anxiety disorder, an eating disorder, an addictive disorder, a sleep disorder, a disease associated with cognitive dysfunction, a neurodegenerative disease, a seizure disorder, a pain disorder; a panic disorder, a disorder of movement, or an obsessive-compulsive disorder.

10) The method according to claim 9, wherein said disease associated with cognitive dysfunction is Alzheimer's disease.

11) The method according to claim 9, wherein said neurodegenerative disease is stroke.

12) The method according to claim 9, wherein said disorder of movement is Parkinson's disease.

13) The method according to claim 9, wherein said seizure disorder is epilepsy.

14) The method according to claim 9, wherein said affective disorder is depression.

15) The method according to claim 5, wherein said compound selectively binds to the 5-HT_{1A} receptor relative to other serotonin receptors.

16) A method according to claim 5 further comprising the steps of:
(c) comparing the radioactive emission of ¹⁸F detected for said subject with standard values;
(d) finding any significant deviation between said radioactive emission of ¹⁸F detected for said subject as compared with said standard values; and
(e) attributing said deviation to a neurological disorder.

17) A method comprising the step of administering to a subject an effective amount of a compound as defined in claim 1 wherein said isotope of fluorine is ¹⁹F.

18) (canceled)

19) A method according to claim 5 wherein said method monitors the effect of treatment of a human or animal body with a drug to combat or treat a condition associated with a neurological disorder before, during and after treatment with said drug.

20) (canceled)

21) (canceled)

22) (canceled)

23) (canceled)

24) (canceled)

25) A method of making the compound of Formula I as defined in claim 1 wherein said isotope of fluorine is ¹⁸F, said method comprising:
(i) reacting a precursor compound of Formula II: 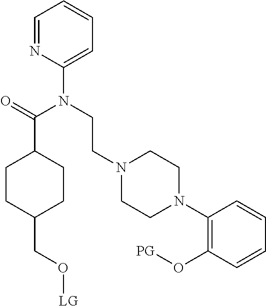
with a source of ¹⁸F fluoride, wherein LG represents hydrogen or a suitable leaving group and PG represents hydrogen or a suitable protecting group; and then,
(ii) removing said protecting group to obtain said compound of Formula I

26) The method according to claim 25, wherein said protecting group is a methoxyethoxymethyl (MEM) group, a methoxymethyl (MOM) group, a t-butyldimethylsilyl (TBDMS) group, a trimethylsilyl (TMS) group or a benzyl group.

27) The method according to claim 25, wherein said leaving group is mesylate, tosylate, brosylate, nosylate, or acyloxy.

28) The method according to claim 25 wherein said method is automated.

29) A kit comprising:
(i) a vessel comprising the precursor compound of Formula II as defined in claim 25; and
(ii) means for eluting the vessel with a source of ¹⁸F⁻.

30) The kit according to claim 28 which further comprises:
(iii) an ion-exchange cartridge for removal of excess ¹⁸F⁻.

31) The kit according to claim 28 which is a cassette suitable for use with an automated synthesis apparatus.

32) (canceled)
